(19) United States
(12) Patent Application Publication
(10) Pub. No.: US 2025/0049857 A1
(43) Pub. Date: Feb. 13, 2025

(54) COMPOSITIONS AND METHODS FOR TREATING KIDNEY DISEASE AND OTHER DISEASE

(71) Applicant: LIVEKIDNEY.BIO LTD, TEL AVIV (IL)

(72) Inventors: MASHA SIMANOVSKY, MAZKERET BATYA (IL); SHIMON AMSELEM, REHOVOT (IL); ALON YAAR, RAMAT HASHARON (IL)

(21) Appl. No.: 18/889,664

(22) Filed: Sep. 19, 2024

Related U.S. Application Data
(63) Continuation-in-part of application No. PCT/IL2024/050527, filed on May 29, 2024.
(60) Provisional application No. 63/505,434, filed on Jun. 1, 2023.

(51) Int. Cl. A61K 35/28; A61K 45/06; A61P 13/12

(52) U.S. Cl. CPC A61K 35/28; A61K 45/06; A61P 13/12

(57) ABSTRACT

Described herein are methods for treatment of kidney disease, comprising administering to a patient in need thereof a cell-derived product to the perirenal adipose tissue (PRAT) of the patient. Optionally, the cell derived product is a plurality of mesenchymal stromal cells. Other diseases may be treated using this method including: a disease of the liver, cardiovascular disease, inflammatory disease, fibrotic disease, autoimmune disease, and disease of a visceral organ.

### CROSS-REFERENCE TO RELATED APPLICATIONS

This is a continuation-in-part of International Patent Application No. PCT/IL2024/050527 filed on May 29, 2024, which in turn claims benefit from U.S. Provisional Patent Application No. 63/505,434, filed Jun. 1, 2023, the contents of which are incorporated by reference herein in their entirety.

### FIELD

Aspects of the invention relate to methods for administration of bio-active substances to the kidney of a patient in need thereof, for treatment of kidney disease, liver disease, and diseases of the cardiovascular system.

### BACKGROUND

Chronic kidney disease (CKD) is evolving from a global public health problem to an ever-growing pandemic, marked by increasing prevalence, high morbidity and high mortality. Glomerular filtration rate (GFR) and albuminuria are proposed as the most accurate indicators of kidney function, with low GFR and increased albuminuria being associated with a high risk of kidney failure requiring renal replacement therapy (RRT) as well as cardiovascular disease, anemia, mineral and bone disorder and other complications.

Regardless of the underlying etiology, CKD inevitably progresses, leading to irreversible nephron loss, end-stage renal disease (ESRD) and/or premature death. Factors that contribute to CKD progression include parenchymal cell loss, chronic inflammation, fibrosis and reduced regenerative capacity of the kidney. Current therapies have limited effectiveness and only delay disease progression, underscoring the need to develop novel therapeutic approaches to either stop or reverse progression.

Mesenchymal stromal (stem) cells (MSCs) are stem cells that demonstrate robust immunoregulatory properties. MSCs can affect many different immune cell subtypes of both the innate and adaptive immune systems. Multiple laboratory and clinical studies over the last three decades have demonstrated that MSCs can suppress proliferation of T cells, B cells, natural killer cells and dendritic cells in a dose-dependent manner. Furthermore, MSCs have the ability to direct macrophages to a more immunotolerant (M2) phenotype that is characterized by alternative activation. Cytokine secretion profiles of T and B-cells are also significantly altered to a less inflammatory and less fibrosis-inducing phenotype by incubation with MSCs, which can further contribute to their observed immunosuppressive properties.

### SUMMARY

Described herein are methods for treatment of kidney disease, comprising administering to a patient in need thereof a cell-derived product to the perirenal adipose tissue (PRAT) of the patient. Optionally, the cell derived product is a plurality of mesenchymal stromal cells. Other diseases may be treated using this method including: a disease of the liver, cardiovascular disease, inflammatory disease, fibrotic disease, autoimmune disease, and disease of a visceral organ.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a photograph showing a mouse kidney into which methylene blue was administered to the PRAT, indicating that the dye remained in the PRAT and delivered to the kidney, but did not escape to the surrounding tissue;

FIGS. 2A-D are graphs indicating levels of various biomarkers in the PRAT and/or kidney tissue of mice in an aristolochic acid (AA) induced model of kidney disease in which umbilical cord mesenchymal stromal cells (UC-MSC) were administered to the PRAT of mice, relative to control mice, in which Arg1 refers to Arginase 1; PBS refers to Phosphate Buffered Saline; and RK refers to right kidney;

FIG. 3 is a graph showing effect of UC-MSC administered to the PRAT of mice on serum creatinine, relative to control mice;

FIGS. 4A, 4B, 4C, and 4D are graphs showing effect of UC-MSC in two different dosages (0.5×10⁶ cells; 1×10⁶ cells), administered to the PRAT of mice, on glucose levels in blood (4A), urine creatinine levels (4B), urine urea levels (4C), and Neutrophil Gelatinase-Associated Lipocalin (Ngal) levels (4D) relative to control mice in high fat diet model, in which HFD refers to high fat diet; “M” refers to million cells; and ND refers to normal diet;

FIGS. 5A-5F are graphs showing effect of UC-MSC in reduction of relative expression of proinflammatory markers Interleukin 6, IL6 (5A), Tumer Necrosis Factor alpha, TNFa (5B), C—C Chemokine 2, CCL2 (5C), and Connexin 43, Cx43 (5D) as well as in reduction of relative expression of pro-fibrotic markers Fibronectin-1, FN1 (5E) and Collagen 1 A1, Col1A1 (5F), in kidney, in two different dosages, after administration to the PRAT of mice fed a high fat diet;

FIGS. 6A-6D are graphs showing effect of UC-MSC in reduction of relative expression of proinflammatory markers (FIGS. 6A, 6B, and 6D, and increasing of relative expression of anti-inflammatory marker (FIG. 6C) in the PRAT, in two different dosages, after administration to the PRAT of mice fed a high fat diet, in which IL1b refers to Interleukin 1b; and KIM1 refers to Kidney Injury Molecule 1.

FIGS. 7A and 7B are graphs showing effect of UC-MSC in reduction of relative expression of pro-inflammatory markers (7A) and pro-fibrotic markers (7B) in liver, in two different dosages, after administration to the PRAT of mice fed a high fat diet.

### DETAILED DESCRIPTION

### Terms

Cellular Product: Cellular Product includes cells which have been shown to be effective in treatment or in alleviating an inflammatory state, or fibrosis. Mesenchymal stromal cells (MSCs) are examples of such a cell. Cellular Product also includes products produced by such cells such as exosomes or extracellular vesicles which are produced by MSCs.

Treatment: Treatment refers to a therapeutic intervention that ameliorates a sign or symptom of a disease or pathological condition after it has begun to develop.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides, are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

The perirenal adipose tissue (PRAT), a component of visceral adipose tissue, has been recently recognized as an important factor that contributes to the maintenance of the cardiovascular system and kidney homeostasis. PRAT is a complex microenvironment consisting of a mixture of white adipocytes and dormant and active brown adipocytes, associated with predipocytes, sympathetic nerve endings, vascular structures, and different types of inflammatory cells. PRAT is considered a unique visceral adipose deposit in terms of its specific anatomical features, regarding vascularization and innervation, in the context of its location in the proximity of the kidney.

The inventors have found that administration to PRAT can be advantageous versus other methods of administration of cellular products, such as Umbilical Cord MSCs (UC-MSCs). Administration via PRAT can be associated with an increased therapeutic effect on kidney disease relative to known methods for administration (such as subcutaneous administration and intravenous administration). In addition, PRAT administration can be associated with a lower dose of cellular product administered relative to conventional methods for administration, without a decrease in activity. Another advantage of PRAT administration is the potential for higher safety by causing less systemic exposure to the administered treatment, when compared to other treatments such as intrarenal administration.

According to a preferred embodiment, UC-MSCs are administered via PRAT to a patient in need thereof. MSCs from other sources, such as placenta, adipose tissue, peripheral blood, bone marrow and others may be used according to an embodiment.

According to an embodiment, the cellular product administered via PRAT to a patient in need thereof is a induced pluripotent stem cells (iPSC). Such iPSC are typically derived from human cells such as skin or blood cells and have been reprogrammed into a pluripotent state.

According to an embodiment, the cellular product administered via PRAT to a patient in need thereof is CAR T cells. CAR T cells are T cells engineered with chimeric antigen receptors to recognize specific cells and bring about their destruction. CAR T therapy's immunomodulatory effects show potential for managing inflammatory conditions by reprogramming immune responses. For example, CAR T-cell therapy has shown promise in treating systemic lupus erythematosus (SLE). By targeting CD19, CAR T therapy depletes B cells, leading to a reduction in autoantibody production. This can result in the reversal of severe manifestations such as glomerulonephritis and other organ damage.

Regulatory T-cell (Treg) therapy has shown promise in treating kidney diseases by modulating the immune system and reducing inflammation. In autoimmune conditions like lupus nephritis or glomerulonephritis, Tregs can help prevent immune cells from attacking kidney tissue, potentially preserving kidney function and preventing further damage.

According to an embodiment, the cellular product administered to a patient in need thereof is a plurality of exosomes or extracellular vesicles (EVs). Optionally, the exosomes or extracellular vesicles are derived from MSCs, preferably UC-MSCs. Exosomes and EVs are secreted by MSCs and may have therapeutic potential similar to their parent cells.

In addition to native exosomes secreted by cells such as MSCs, exosomes and EVs containing an active pharmaceutical or biological ingredient may be prepared and administered to a patient in need thereof. Exogenous drug loading methods work passively by the association of drugs with the exosomes or extracellular vesicles (EV) lipid bilayer membrane after incubation, by attaching therapeutics to the exosome/EV surface and by mechanical or chemical techniques to transiently open the exosome/EV membrane to allow diffusion of compounds into the vesicle. The most common approaches to temporarily permeabilize the membrane include sonication, electroporation, saponin treatment and passive incubation. According to an embodiment, an exosome/EV contains an active pharmaceutical ingredient selected from the group consisting of a sodium-glucose cotransporter-2 (SGLT2) inhibitor, angiotensin receptor blocker (ARB), angiotensin-converting-enzyme (ACE) inhibitor and a mineralocorticoid receptor antagonist. Exemplary SGLT2 inhibitors include but are not limited to: bexagliflozin, canagliflozin, dapagliflozin, empagliflozin and ertugliflozin. Exemplary ARBs are selected from the group consisting of: candesartan, valsartan, irbesartan, telmisartan, eprosartan, olmesartan, azilsartan and fimasartan. Exemplary ACE inhibitors are selected from the group consisting of: captopril, enalapril, lisinopril, benazepril, fosinopril, quinapril, ramipril, perindopril, moexipril, trandolapril, alacepril, zofenopril, imidapril and cilazapril. Exemplary mineralocorticoid receptor antagonists are selected from the group consisting of: mexrenone, finerenone, canrenone, eplerenone and spironolactone. The free or drug-loaded exosomes can be formulated for perirenal administration as the following sterile injectable dosage forms (a) hydrophilic formulations containing buffer, electrolytes to adjust the isotonicity, sugars, glycols, aminoacids, antioxidants, preservatives, polymeric stabilizers, surfactants, peptides, albumin (b) hydrophilic hydrogels containing cellulose-derived polymers like methyl cellulose, ethyl cellulose, hydroxypropylmethyl cellulose, hyaluronic acid, polysaccharides, gums, thermosensitive polymers like poloxamers, polyvinyl alcohol, (c) lipid-based formulations containing super-refined vegetable oils and fats, phospholipids, polyethylene-glycol derivatives, glycerides.

According to an embodiment, the amount of cellular product administered per PRAT administration is an amount effective to show an improvement in a symptom or marker of kidney disease. In the case of administration of MSC, the amount administered is preferably up to 500 million cells administered. In the case of exosomes/EVs, the amount administered is preferably between 1×10⁹ to 1×10¹² particles per administration.

Various types of kidney disease may be treated according to embodiments. According to an embodiment, the kidney disease is chronic kidney disease. Optionally, the kidney disease is end stage kidney disease. In addition to treating kidney disease, the methods described herein may be used in treatment of kidney disease co-morbidities. Such co-morbidities include, but are not limited to: high blood pressure, diabetes and obesity. Optionally, a patient in need thereof suffering from kidney disease may be treated using cellular product administered through the PRAT administration, in cases wherein the patient is suffering from a disease selected from the group consisting of: Fabry disease, cystinosis, glomerulonephritis, IgA nephropathy, lupus nephritis, systemic lupus erythematosus, atypical hemolytic uremic syndrome, kidney fibrosis, nephrogenic systemic fibrosis, Focal Segmental Glomerulosclerosis (FSGS), APOLI Nephropathy or polycystic kidney disease.

According to an embodiment, a PRAT administration may be performed using ultrasound guided administration or laparoscopy. Optionally, the PRAT administration is performed using augmented reality.

Treatments described herein may contribute to an improvement in disease state. Optionally, the improvement in disease state may be evident by decrease in inflammation or in fibrosis biomarkers, slowing deterioration of, maintenance of or improvement in GFR, reducing serum and urine creatinine, urea, N-gal as well as blood glucose.

Described herein, according to some embodiments, are compositions for use in treating disease, according to the following methods: A method for treatment of a subject suffering from kidney disease comprising administering to the perirenal adipose tissue of the subject a cellular product in an amount effective to show an improvement in a symptom or marker of kidney disease in the subject. Optionally, the cellular product comprises a plurality of mesenchymal stromal cells (MSC). Optionally, the MSC is of umbilical cord origin. Optionally, the cellular product comprises an extracellular vesicle or an exosome. Optionally, the extracellular vesicle or exosome comprises an active ingredient selected from the group consisting of a sodium-glucose cotransporter-2 (SGLT2) inhibitor, angiotensin receptor blocker (ARB), angiotensin-converting-enzyme (ACE) inhibitor, and a mineralocorticoid receptor antagonist. Optionally, the amount of MSC administered is between 10-100 million cells per ml. Optionally, the amount of MSC administered is up to 500 million cells per administration. Optionally, the subject suffers from a disease selected from the group consisting of: chronic kidney disease, end stage kidney disease, Fabry disease, cystinosis, glomerulonephritis, IgA nephropathy, lupus nephritis, systemic lupus erythematosus, atypical hemolytic uremic syndrome, kidney fibrosis, nephrogenic systemic fibrosis, Focal Segmental Glomerulosclerosis (FSGS), APOLI Nephropathy, and polycystic kidney disease. Optionally, the subject suffers from a kidney disease co-morbidity selected from the group consisting of: high blood pressure, diabetes, and obesity.

Further described herein, according to an embodiment is a method for treatment of a subject suffering from cardiovascular disease comprising administering to the perirenal adipose tissue of the subject a cellular product in an amount effective to show an improvement in a symptom or marker of cardiovascular disease in the subject.

Further described herein according to an embodiment, is a method for treatment of a subject suffering from inflammatory or fibrotic disease of the liver comprising administering to the perirenal adipose tissue of the subject a cellular product in an amount effective to show an improvement in a symptom or marker of inflammatory or fibrotic disease of the liver in the subject.

Further described herein according to an embodiment, is a method for treatment of a subject suffering from an autoimmune disease, comprising administering to the perirenal adipose tissue of the subject a cellular product in an amount effective to show an improvement in a symptom or marker of autoimmune disease in the subject.

Further described herein, according to an embodiment, is a method for treatment of a subject suffering from a disease of a visceral organ, comprising administering to the perirenal adipose tissue of the subject a cellular product in an amount effective to show an improvement in a symptom or marker of disease of a visceral organ in the subject.

### EXAMPLES

The following examples are provided to illustrate certain particular features and/or embodiments. These examples should not be construed to limit the disclosure to the particular features or embodiments described.

### Example 1: Evaluation of Perirenal Adipose Tissue (PRAT) Administration in Mice in an Aristolochic Acid (AA) Induced Model of Kidney Disease

An in vivo test of PRAT administration to mice was performed in which PRAT administration is performed. A single administration of human umbilical cord-derived Mesenchymal stromal cells (UC-MSC) via PRAT was performed on mice in an AA-induced nephropathy model.

Aristolochic acid is a nephrotoxin derived from the plant of the Aristolochiaceae family. A model is performed using C57BL6 male mice, aged 16 weeks. The mice underwent four once-weekly intraperitoneal (IP) injections of 4 milligrams per kilogram (mg/kg) of AA on days 1, 7, 14, and 21 to induce a disease-like state resembling acute kidney injury. Treatment was administered on day 24, 3 days after the last AA injection, in the form of a 50 microliter injection to the PRAT region of the right kidney.

Four groups of mice were used in the study. Group 1 received a single PRAT injection of UC-MSCs in an amount of 5×10⁶ cells in PBS buffer. Group 2 received a single PRAT injection of PBS to the PRAT. Group 3 received AA alone, without a treatment, and serves as a control. Group 4 was a naïve group of mice for which a disease state is not induced.

Serum was collected and assessed for creatinine and various biomarkers. Mice from all groups were observed until day 54, which is the end of the study. At the end of the study, animals were sacrificed and PRAT and associated kidneys were collected. Fibrosis and kidney inflammation were determined by histopathological examination (H&E and Masson trichrome staining). Expression of inflammation and fibrosis markers was determined by RT-qPCR.

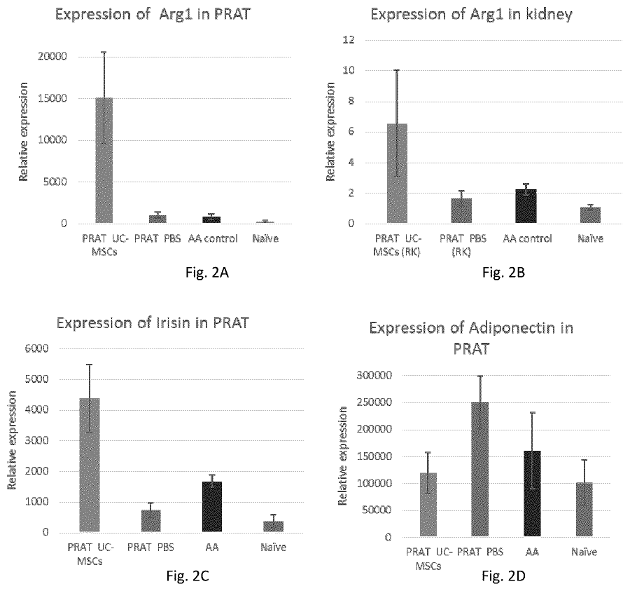

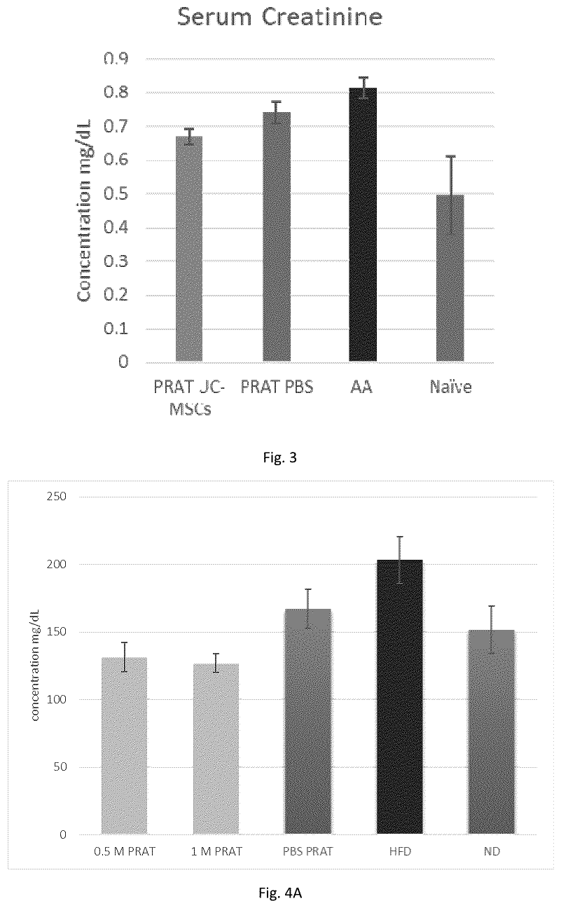

Administration of UC-MSCs into PRAT resulted in changes in the expression of adipokines within the PRAT, as well as in levels of tissue and serum biomarkers. An upregulation of Arg1, a marker of the M2 state, anti-inflammatory, pro-proliferative and healing-supportive phenotype, both in the PRAT and kidneys was demonstrated, as seen in FIGS. 2A and 2B respectively. In addition, an increase in relative expression of Irisin alongside a decrease in the relative expression of Adiponectin in PRAT was demonstrated (FIGS. 2C and 2D). Irisin is released from myocytes during physical activity, and acts as a link between muscles and other tissues and organs. The two N-glycan molecules, which constitute a significant part of the Irisin glycoprotein, regulate the browning of adipocytes, which is the most important function of Irisin. Studies have confirmed the multifunctional role of Irisin and the beneficial effects of this molecule on body homeostasis. Irisin reduces systemic inflammation, maintains the balance between resorption and bone formation, and modulates metabolic processes and the functioning of the nervous system. It suppresses the expression and release of pro-inflammatory cytokines in obese individuals and attenuates inflammation in adipose tissue. In addition, the literature indicates that higher levels of Irisin can have a beneficial effect on the cardiovascular system. Systemically, PRAT administration of UC-MSCs induced a modest decrease in serum creatinine levels (FIG. 3). Histological evaluation of kidney and PRAT revealed the absence of toxic effects in kidneys following PRAT injection of UC-MSCs, supporting the safety of this approach.

Administration of UC-MSC into the PRAT induced both a local anti-inflammatory effect (induction of Arg1 in PRAT and kidney) as well as endocrinal effects in the PRAT (induction of Irisin and reduction of Adiponectin) which could be indicative of a more general systemic anti-inflammatory metabolic state. In addition, the modest reduction of serum creatinine supports the induction of a healing process in the kidneys resulting from the anti-inflammatory paracrine effects of UC-MSC injected into PRAT.

### Example 2A: Administration of Methylene Blue (MB) to the Perirenal Adipose Tissue of Healthy Mice

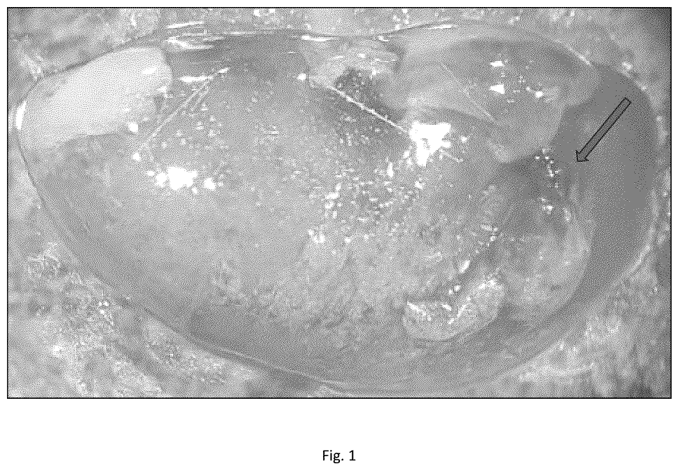

A 1% solution of Methylene Blue was injected into the perirenal adipose tissue of healthy mice. After 24 hours, mice were euthanized, and the kidneys were sectioned sagittally for visual inspection, as shown in FIG. 1. Upon examination, the presence of blue coloration was observed exclusively in the renal tissue, as indicated by the blue arrow, and in the PRAT, but not in the surrounding tissue. This indicates that delivery specifically to the PRAT can be used for treatment and delivery to the kidney.

### Example 2B: Administration of Human UC-MSCs to Mice

Using the procedure described in Example 2A, 2×10⁶ human UC-MSCs were injected into the PRAT of healthy mice. After 1 week of observation, the mice were found to be in good condition.

### Example 3: Therapeutic Effect of UC-MSC Via PRAT in an Obesity Related Nephropathy Model in C57BL6 Mice

Single administration of 0.5×10⁶ or 1×10⁶ human umbilical cord-derived Mesenchymal Stromal Cells (UC-MSC), via perirenal adipose tissue (PRAT) injection, in a High Fat Diet (HFD) obesity related nephropathy model in C57BL6 mice was performed.

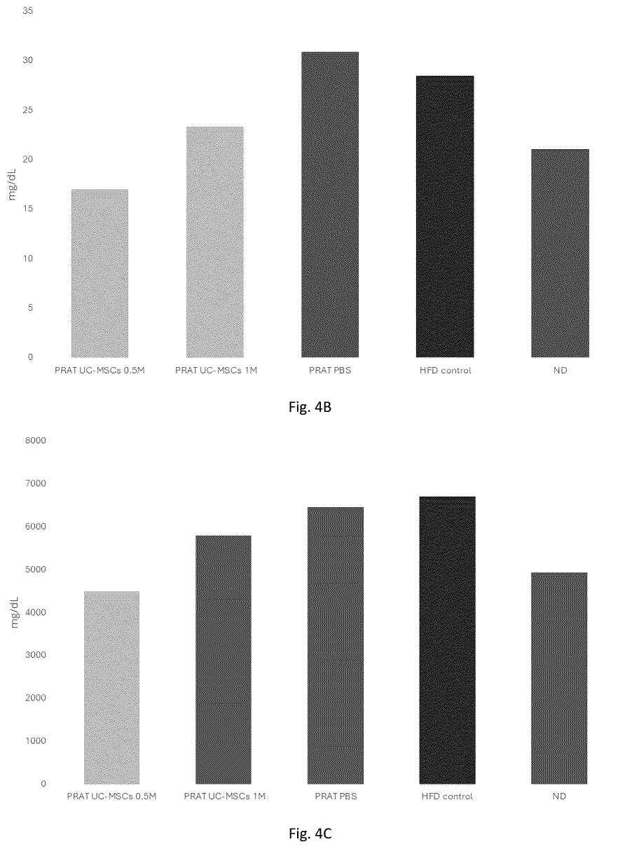

Male C57BL/6J mice, aged 8 weeks, were fed a high-fat diet (HFD; 60% fat) for 8 weeks. Age-matched male C57BL/6J mice were fed a standard laboratory normal diet (ND; 14% fat) as a control group. At week 8, either 0.5×10⁶ or 1×10⁶ UC-MSCs, suspended in PBS, or PBS alone was injected into the right PRAT. The mice from the HFD group continued on the HFD for an additional 6 weeks. At week 14, the mice were sacrificed and their kidneys, PRAT, liver and heart samples were collected for analysis. Administration of 0.5×10⁶ and/or 1×10⁶ UC-MSC intra-PRAT induced both systemic and local tissue-level effects. As illustrated in FIG. 4A, HFD mice experienced an increase (from approximately 150 mg/dL to 200 mg/dL, a ˜30% increase) in their blood glucose levels as compared to normal diet mice. Administration of both dose levels of UC-MSC intra-PRAT lowered blood glucose levels to approximately 120-130 mg/dL, 20-30 units below control baseline (ND, Normal Diet).

Moreover, HFD mice demonstrated increased urinary creatinine and urea levels, indicating a reduction of normal kidney function. As shown in FIGS. 4B and 4C, administration of 0.5×10⁶ UC-MSC intra-PRAT reduced the levels of these metabolic markers below control levels. Neutrophil Gelatinase-Associated Lipocalin (Ngal), a 21-kD protein of the lipocalin superfamily, is a component of innate immunity to bacterial infection and is expressed by immune cells, hepatocytes and renal tubular cells in various disease states. Ngal is protease resistant and thus may be easily detected in the urine. This marker has emerged as a potential biomarker for diabetic nephropathy due to its association with renal injury and its ability to provide early indications of kidney damage. As illustrated in FIG. 4D, both doses of UC-MSC reduced elevated Ngal to control levels.

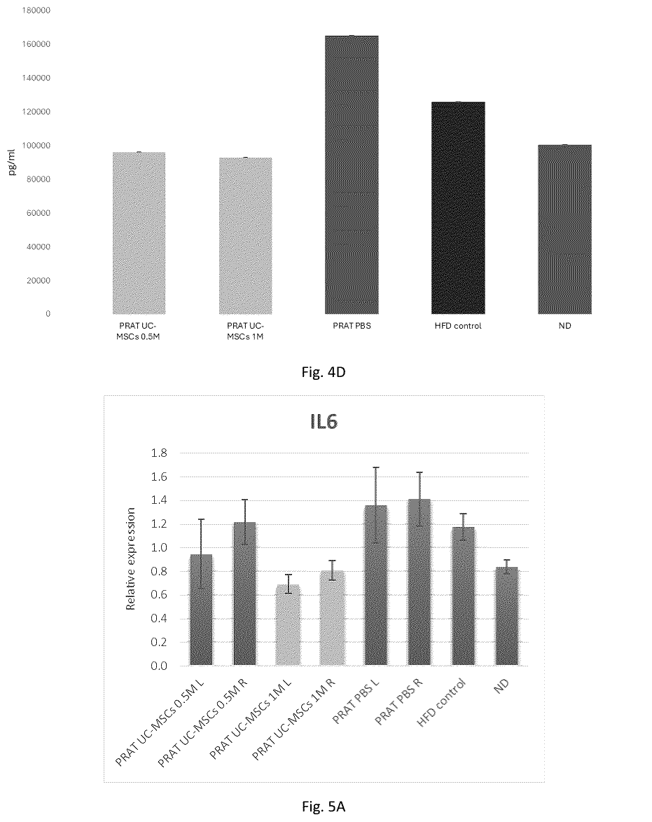

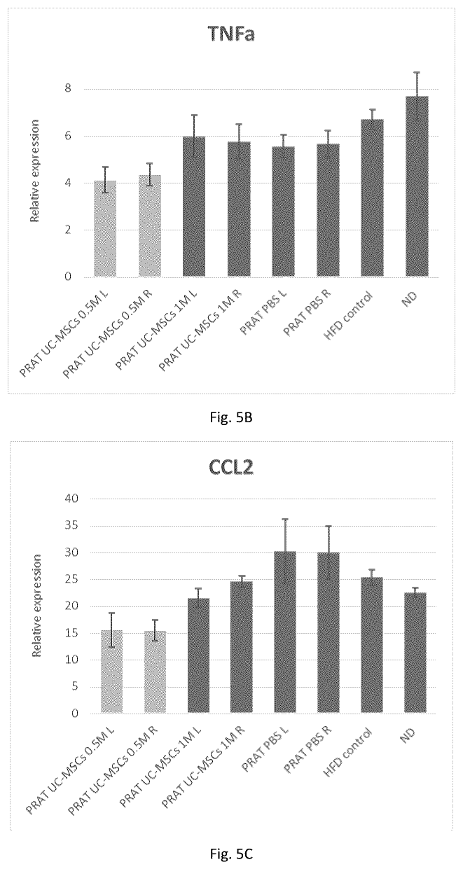

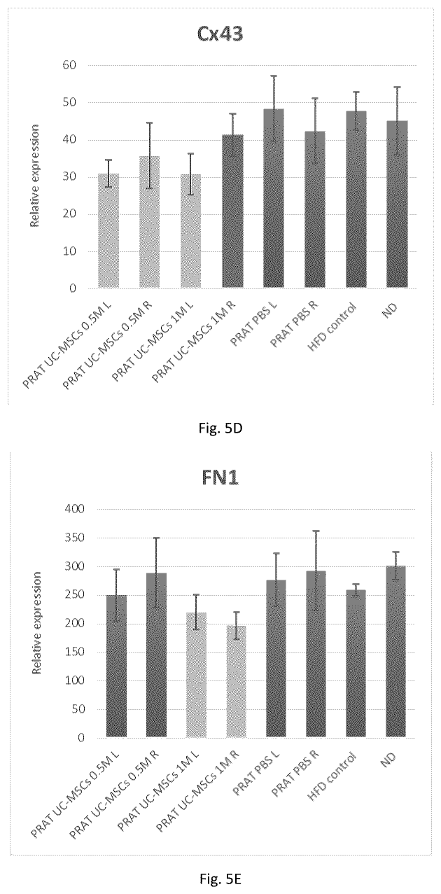

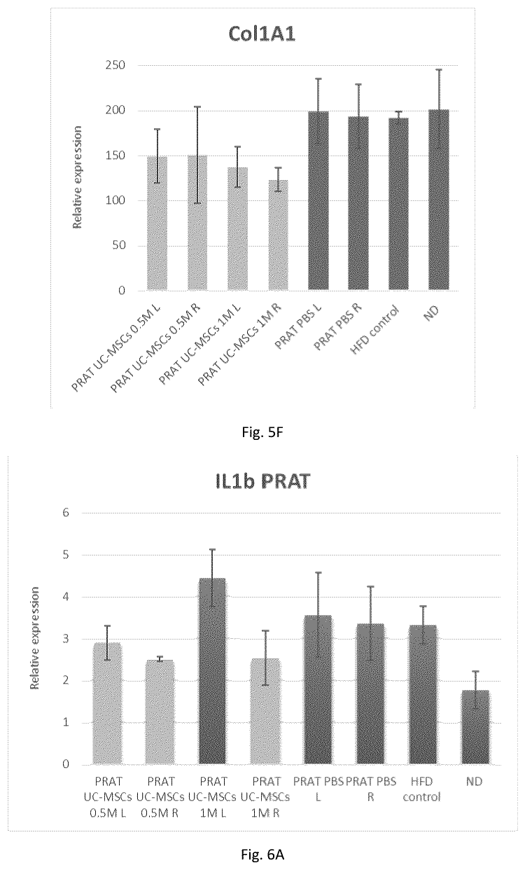

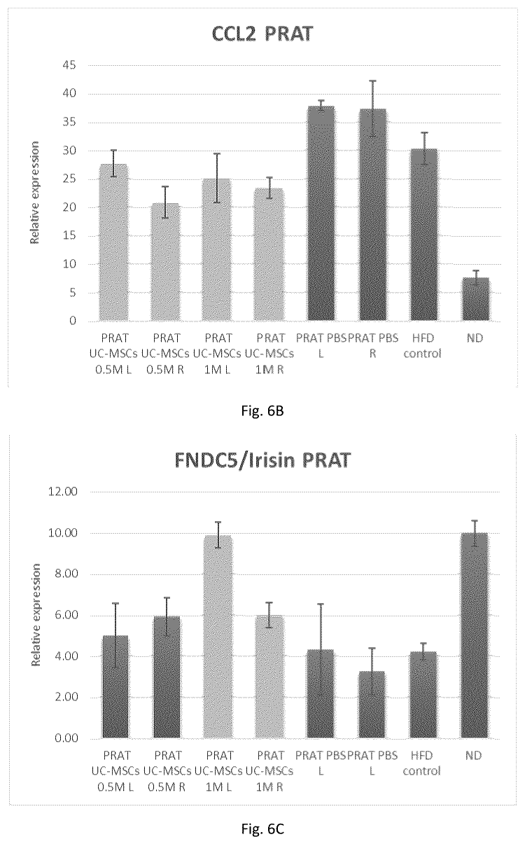

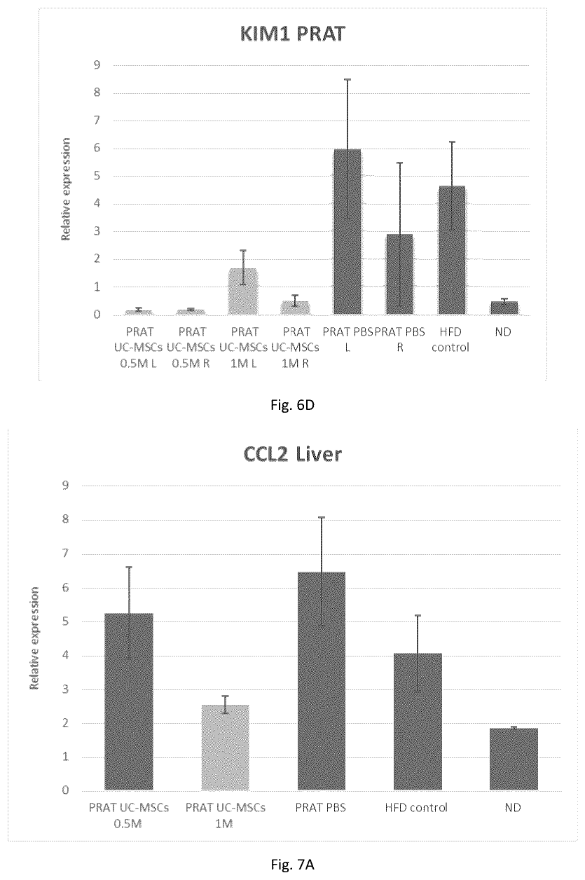

Furthermore, UC-MSC administration intra-PRAT caused a reduction in relative expression of pro-inflammatory markers IL6, TNFa, C—C Chemokine 2 (CCL2) and Connexin 43 (Cx43) as well as pro-fibrotic markers fibronectin-1 (FN1) and Collagen 1A1 (Col1A1), as depicted in FIGS. 5A-F. In the PRAT itself, ipsilateral (Right kidney, indicated by “R”) and/or contralateral (left kidney, indicated by “L”) UC-MSC administration triggered a reduction in relative expression of IL1b (FIG. 6A) and CCL2 (pro-inflammatory markers, FIG. 6B) as well as a sharp reduction in relative expression of Kidney Injury Molecule 1 (KIM1, FIG. 6D), a transmembrane glycoprotein expressed by proximal tubular cells recognized as an early, sensitive and specific biomarker for kidney injury. In contrast, UC-MSC administration intra-PRAT induced an increase in the relative expression of the anti-inflammatory factor Irisin, as illustrated in FIG. 6C.

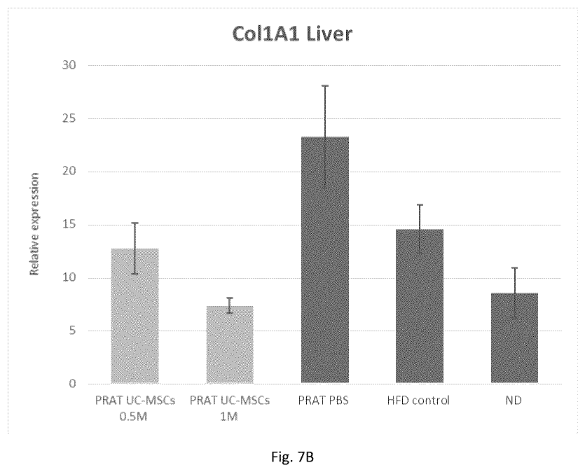

The relative expression of CCL2 and Col1A1 in the liver were also examined. As shown in FIGS. 7A and 7B, UC-MSC administered intra-PRAT caused a reduction in the relative expression of these two markers, thus further supporting the systemic anti-inflammatory and anti-fibrotic effects produced by this method of administration, even in non-kidney organs such as the liver.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treatment of a subject suffering from kidney disease comprising administering to the perirenal adipose tissue of the subject a cellular product in an amount effective to show an improvement in a symptom or marker of kidney disease in the subject.

2. The method according to claim 1 wherein the cellular product comprises a plurality of mesenchymal stromal cells (MSC).

3. The method according to claim 2 wherein the MSC is of umbilical cord origin.

4. The method according to claim 2 wherein the cellular product comprises an extracellular vesicle or an exosome.

5. The method according to claim 4 wherein the extracellular vesicle or exosome comprises an active ingredient selected from the group consisting of a sodium-glucose cotransporter-2 (SGLT2) inhibitor, angiotensin receptor blocker (ARB), angiotensin-converting-enzyme (ACE) inhibitor, and a mineralocorticoid receptor antagonist.

6. The method according to claim 2 wherein the amount of MSC administered is between 10-100 million cells per ml.

7. The method according to 1 wherein the amount of MSC administered is up to 500 million cells per administration.

8. The method according to claim 1 wherein the subject suffers from a disease selected from the group consisting of: chronic kidney disease, end stage kidney disease, Fabry disease, cystinosis, glomerulonephritis, IgA nephropathy, lupus nephritis, systemic lupus erythematosus, atypical hemolytic uremic syndrome, kidney fibrosis, nephrogenic systemic fibrosis, Focal Segmental Glomerulosclerosis (FSGS), APOLI Nephropathy, and polycystic kidney disease.

9. The method according to claim 1 wherein the subject suffers from a kidney disease co-morbidity selected from the group consisting of: high blood pressure, diabetes, and obesity.
